(19) United States
(12) Patent Application Publication
(10) Pub. No.: US 2026/0235574 A1
(43) Pub. Date: Aug. 13, 2026

(54) SOIL CONTAMINATION EVALUATION METHOD

(71) Applicant: SHIMIZU CORPORATION, Tokyo (JP)

(72) Inventor: Yoji AOKI, Tokyo (JP)

(73) Assignee: SHIMIZU CORPORATION, Tokyo (JP)

(21) Appl. No.: 19/471,722

(22) PCT Filed: Apr. 10, 2024
(86) PCT No.: PCT/JP2024/014512

§ 371 Date: Oct. 2, 2025

(30) Foreign Application Priority Data
Apr. 17, 2023 (JP) 2023-067355

(51) Int. Cl. G01N 33/24; G01N 1/22

(52) U.S. Cl. CPC G01N 33/24; G01N 1/2294

(57) ABSTRACT

Disclosed a soil contamination evaluation method including a step (I) of collecting soil gas from soil and a step (II) of estimating concentrations of dioxins contained in the soil from a content of an organic compound contained in the soil gas.

### TECHNICAL FIELD

The present invention relates to a soil contamination evaluation method. Priority is claimed on Japanese Patent Application No. 2023-067355, filed Apr. 17, 2023, the content of which is incorporated herein by reference.

### BACKGROUND ART

In a case in which a purification treatment is performed for soil contaminated with contaminants such as heavy metals, volatile organic compounds, and oil, in the related art, a method in which a treatment target area is equally divided into division units and a boring investigation, a gas analysis, or the like is performed for each division unit is known. A contamination status in each division unit is checked through the boring investigation, the gas analysis, or the like, and a purification treatment is performed for soil in a division unit whose contamination status exceeds a reference value. An investigation for finely divided division units becomes more time-consuming and costly as the treatment target area becomes larger.

To solve such a problem, for example, Patent Document 1 proposes a soil contamination evaluation method in which a general investigation of the contaminants is performed, a three-dimensional structure of the soil is analyzed on the basis of the results of the general investigation, and the behavior of the contaminants in the soil is estimated. According to the invention of Patent Document 1, an attempt is made to efficiently select the area to be purified.

### CITATION LIST

### Patent Document

Patent Document 1: Japanese Unexamined Patent Application, First Publication No. 2003-94036

### SUMMARY OF INVENTION

### Technical Problem

Among various contaminants, dioxins are non-volatile and stable organochlorine compounds that are of concern due to their effects on the human body. For this reason, it is difficult to check the status of contamination with dioxins using a gas analysis as described in Patent Document 1.

The present invention has been made in view of the above circumstances, and an object of the present invention is to provide a soil contamination evaluation method that can simply and easily investigate the status of contamination of soil contaminated with dioxins.

### Solution to Problem

To solve the above problem, the present invention has the following aspects.

[1]A soil contamination evaluation method including a step (I) of collecting soil gas from soil and a step (II) of estimating concentrations of dioxins contained in the soil from a content of an organic compound contained in the soil gas.

[2] The soil contamination evaluation method according to [1], wherein in the step (II), it is determined that the soil is contaminated with the dioxins in high concentrations when the content of the organic compound is 0.1 mg/kg or more.

[3] The soil contamination evaluation method according to [1] or [2], wherein the step (1) has a treatment of adsorbing the organic compound to an adsorbent and a treatment of desorbing the organic compound from the adsorbent to which the organic compound has been adsorbed, and wherein the step (II) has a treatment of identifying a type of the desorbed organic compound.

[4] The soil contamination evaluation method according to any one of [1] to [3], wherein the step (I) has a treatment of heating the soil.

[5] The soil contamination evaluation method according to any one of [1] to [4], wherein the organic compound includes one or more selected from 2,4-dichlorophenol, 2,4-dichloroanisole, 2,4,5-trichlorophenol, and 2,4,5-trichloroanisole.

### Advantageous Effects of Invention

According to a soil contamination evaluation method of the present invention, it is possible to simply and easily investigate the status of contamination of soil contaminated with dioxins.

### BRIEF DESCRIPTION OF DRAWINGS

FIG. 1 A flowchart illustrating a soil contamination evaluation method according to one embodiment of the present invention.

FIG. 2 A schematic view illustrating an example of a step (I) according to one embodiment of the present invention.

FIG. 3 A graph illustrating a correlation between a concentration of an organic compound and a concentration of dioxins, which are detected in soil.

FIG. 4 A schematic view illustrating an example of a step (I) according to another embodiment of the present invention.

### DESCRIPTION OF EMBODIMENTS

### <Soil Contamination Evaluation Method>

A soil contamination evaluation method of the present invention includes a step (I) of collecting soil gas from soil and a step (II) of estimating concentrations of dioxins contained in the soil from a content of an organic compound contained in the soil gas.

Hereinafter, a soil contamination evaluation method according to one embodiment of the present invention will be described with reference to the drawings.

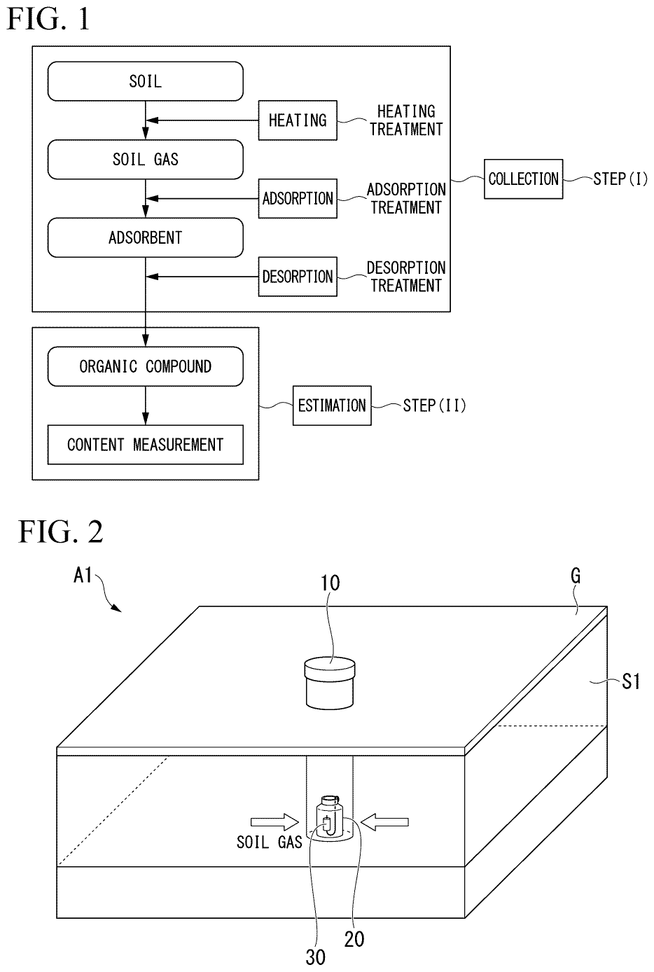

As shown in FIG. 1, the soil contamination evaluation method of the present embodiment includes a step (I) and a step (II). Furthermore, the step (I) of the soil contamination evaluation method of the present embodiment includes a treatment of heating soil (a heating treatment), a treatment of adsorbing an organic compound to an adsorbent (an adsorption treatment), and a treatment of desorbing the organic compound from the adsorbent (a desorption treatment).

Hereinafter, each step will be described in detail.

### <Step (I)>

The step (I) is a step of collecting soil gas from the soil.

In this specification, the “soil gas” refers to air in the soil. The soil gas has a lower oxygen concentration and a higher carbon dioxide concentration than atmospheric air due to the consumption of oxygen and the release of carbon dioxide by microorganisms and plant roots. By collecting and analyzing the soil gas, it is possible to indirectly find microbial metabolism and components contained in the soil. The soil gas collected from soil contaminated with a defoliant contains an organic compound derived from the defoliant.

In this specification, the “defoliant” refers to one of pesticides used to kill plants, also known as a herbicide. The defoliant in this specification includes 2,3,7,8-tetrachlorodibenzo-1,4-dioxin (TCDD) which is one of the dioxins.

In this specification, the “dioxins” is a general term for polychlorinated dibenzoparadioxins (PCDDs), polychlorinated dibenzofurans (PCDFs), and dioxin-like polychlorinated biphenyls (DL-PCBs). DL-PCBs are polychlorinated biphenyls (PCBs) that have toxicity specific to the dioxins. The toxicity of the dioxins is expressed using toxic equivalents (TEQ) based on the toxicity of the 2,3,7,8-tetrachlorodibenzo-1,4-dioxin (TCDD).

One embodiment of the step (I) will be described with reference to the drawing.

As shown in FIG. 2, when collecting the soil gas, a collection hole is drilled from a ground surface G into soil S1 for an arbitrary treatment target area A1 (for example, an area of 10 m×10 m), and a protective tube 10 is buried. A collection container 20 is installed inside the protective tube 10, and an adsorbent 30 is installed inside the collection container 20 not to come into contact with the inner surface of the collection container 20. The size of the collection hole is not particularly limited, and the diameter of the collection hole is preferably, for example, 50 to 75 mm. The depth of the collection hole is preferably, for example, 0.8 to 1 m.

The protective tube 10 is preferably a tube made of a material that does not adsorb an investigation target substance, such as a stainless steel tube or an aluminum tube. The protective tube 10 preferably has an opening portion on a bottom surface or a lower side surface. The protective tube 10 has the opening portion at the bottom surface or the lower side surface, and thus the soil gas can be introduced into the inside of the protective tube 10. It is preferable that 50 cm or more of the upper portion of the protective tube 10 be a non-perforated tube. By making 50 cm or more of the upper portion of the protective tube 10 a non-perforated tube, it is possible to prevent the atmospheric air from entering the inside of the protective tube 10, thereby further improving the accuracy of an analysis of the components contained in the soil gas. It is preferable that the head of the protective tube 10 can be tightly plugged with a rubber plug, a packer, or the like. By tightly plugging the head of the protective tube 10 with a rubber plug, a packer, or the like, it is possible to prevent the atmospheric air from entering the inside of the protective tube 10, thereby further improving the accuracy of an analysis of the components contained in the soil gas. The size of the protective tube 10 is not particularly limited, and it is preferable that the size be slightly smaller than the diameter of the collection hole and longer than the depth of the collection hole.

The collection container 20 is placed inside the protective tube 10. The material of the collection container 20 is preferably one that does not affect the analysis results of the soil gas through chemical reactions, adsorption reactions, and the like and is not easily corroded by substances contained in the soil gas. Examples of the material of the collection container 20 include glass, silicone resin, fluororesin, and the like.

The inner diameter of the collection container 20 only needs to be smaller than the diameter of the protective tube 10 and can be determined appropriately taking into consideration the easiness of inflow of the soil gas, the strength of the collection container 20, the easiness of cleaning, and the like. The length of the collection container 20 only needs to be shorter than the length of the protective tube 10 and is preferably long enough to be inserted close to the opening portion of the protective tube 10.

When the collection container 20 that has been used once is to be reused, it is preferable to wash it thoroughly before use. Examples of a method for cleaning the collection container 20 include water cleaning, heating cleaning (heating removal), and the like.

The adsorbent 30 is not particularly limited as long as it can adsorb the organic compound contained in the soil gas. Examples of the adsorbent 30 include silica having fine holes, activated carbon having fine holes, a porous polymer, and a zeolite having fine holes.

Examples of the organic compound contained in the soil gas include organic compounds derived from the defoliant, such as an aliphatic hydrocarbon, an aromatic hydrocarbon, phenol, anisole, and halides thereof.

Examples of the aliphatic hydrocarbon include undecane, dodecane, pentadecane, hexadecane, octadecane, and the like.

Examples of the aromatic hydrocarbon include benzene, toluene, xylene, naphthalene, and the like.

Examples of other organic compounds contained in the soil gas include an organic siloxane, limonene, an aliphatic carboxylic acid, acetone, and the like.

### (Heating Treatment)

In the soil contamination evaluation method of the present embodiment, the step (I) preferably has a treatment of heating the soil (hereinafter also referred to as a “heating treatment”). Since the step (I) has the heating treatment, it is possible to detect a larger amount of the organic compound contained in the soil gas.

The temperature in the heating treatment (hereinafter also referred to as a “heating temperature”) is, for example, preferably 30° C. or higher and lower than 100° C., more preferably 40° C. or higher and 90° C. or lower, and even more preferably 50° C. or higher and 70° C. or lower. When the heating temperature is equal to or higher than the lower limit value, it is possible to further increase the detection amount of the organic compound, and it is possible to further improve the accuracy of determining whether or not the soil is contaminated with the dioxins. When the heating temperature is less than the upper limit value, it is possible to suppress the generation of water vapor. In addition, when the heating temperature is less than the upper limit value, it is possible to suppress the deterioration of the soil.

The time in the heating treatment (hereinafter also referred to as a “heating time”) is, for example, preferably 5 to 120 minutes, more preferably 10 to 90 minutes, and even more preferably 20 to 60 minutes. When the heating time is equal to or higher than the lower limit value, it is possible to further increase the detection amount of the organic compound, and it is possible to further increase the accuracy of determining whether or not the soil is contaminated with the dioxins. When the heating time is equal to or less than the upper limit value, it is possible to suppress the deterioration of the soil.

A heating treatment method is not particularly limited, and examples thereof include a method of burying a heater in the soil and heating the soil. Examples of a method for heating the soil by burying a heater include a method in which a heater is installed in the collection hole to directly heat the soil, a method in which a heater is buried in the soil around the collection hole to indirectly heat the soil, and the like.

### (Adsorption Treatment)

The step (I) preferably has a treatment of adsorbing the organic compound to an adsorbent (hereinafter also referred to as an “adsorption treatment”). Since the step (I) has the adsorption treatment, it is possible to more efficiently investigate the content of the organic compound contained in the soil gas. That is, the soil contamination evaluation method of the present embodiment has the adsorption treatment, and thus it is possible to more simply and easily investigate the status of contamination of the soil contaminated with the dioxins.

The adsorbent is not particularly limited, and examples thereof include the adsorbent 30 described above.

The treatment time in the adsorption treatment (hereinafter also referred to as an “adsorption time”) is, for example, preferably 5 to 120 minutes, more preferably 10 to 90 minutes, and even more preferably 20 to 60 minutes. When the adsorption time is equal to or higher than the lower limit value, it is possible to further increase the detection amount of the organic compound, and it is possible to further improve the accuracy of determining whether or not the soil is contaminated with the dioxins. When the adsorption time is equal to or less than the upper limit value, it is possible to sufficiently adsorb the soil gas, and it is possible to further improve the evaluation efficiency.

The temperature in the adsorption treatment (hereinafter also referred to as an “adsorption temperature”) is, for example, preferably 30° C. or higher and lower than 100° C., more preferably 40° C. or higher and 90° C. or lower, and even more preferably 50° C. or higher and 70° C. or lower. When the adsorption temperature is equal to or higher than the lower limit value, it is possible to further increase the detection amount of the organic compound, and it is possible to further improve the accuracy of determining whether or not the soil is contaminated with the dioxins. When the adsorption temperature is equal to or lower than the upper limit value, it is possible to further increase the amount of the organic compound adsorbed to the adsorbent.

### (Desorption Treatment)

The step (I) preferably has a step of desorbing the organic compound from the adsorbent to which the organic compound has been adsorbed (hereinafter also referred to as a “desorption treatment”). Since the step (I) has the desorption treatment, it is possible to more simply and easily measure the content of the organic compound contained in the soil gas. That is, the soil contamination evaluation method of the present embodiment has the desorption treatment, and thus it is possible to more simply and easily investigate the status of contamination of the soil contaminated with the dioxins.

Examples of a method for desorbing the organic compound from the adsorbent to which the organic compound has been adsorbed include a heating method, a solvent extraction method, and the like.

In the case of the heating method, the treatment temperature in the desorption treatment (hereinafter also referred to as a “desorption temperature”) is, for example, preferably 150° C. to 350° C., more preferably 200° C. to 300° C. and even more preferably 220° C. to 280° C. When the desorption temperature is equal to or higher than the lower limit value, it is possible to further increase the desorption amount of the organic compound, and it is possible to further improve the accuracy of determining whether or not the soil is contaminated with the dioxins. When the desorption temperature is equal to or less than the upper limit value, it is possible to suppress the deterioration of the adsorbed components.

In the case of the heating method, the treatment time in the desorption treatment (hereinafter also referred to as a “thermal desorption time”) is, for example, preferably 0.5 to 30 minutes, more preferably 1 to 20 minutes, and even more preferably 1.5 to 10 minutes. When the heating desorption time is equal to or higher than the lower limit value, it is possible to further increase the detection amount of the organic compound, and it is possible to further improve the accuracy of determining whether or not the soil is contaminated with the dioxins. When the thermal desorption time is equal to or less than the upper limit value, it is possible to sufficiently desorb the organic compound, and it is possible to further improve the evaluation efficiency.

In the solvent extraction method, for example, dichloromethane, acetone, methanol, ethanol, hexane, diethyl ether, acetonitrile, toluene, or the like is used as a solvent, and it is possible to desorb the organic compound from the adsorbent and to extract the organic compound into the solvent. In this case, since the extraction rate of the substance varies depending on the type of solvent, it is desirable to select in advance a solvent suitable for the substance to be extracted. In general, in a case in which the extraction time is short and the amount of the solvent used is small, the extraction rate decreases.

### <Step (II)>

The step (II) is a step of estimating a concentration of the dioxins contained in the soil from the content of the organic compound contained in the soil gas.

The defoliant of the present embodiment includes the TCDD. The TCDD is a non-volatile organic compound and is therefore not detectable from the soil gas.

However, 2,4-dichlorophenoxyacetic acid (2,4-D) which is one of the main components of the defoliant is decomposed in the soil by microorganisms and the like into 2,4-dichlorophenol and 2,4-dichloroanisole.

Similarly, 2,4,5-trichlorophenoxyacetic acid (2,4,5-T) which is one of the main components of the defoliant is decomposed in the soil by microorganisms and the like into 2,4,5-trichlorophenol and 2,4,5-trichloroanisole.

These decomposed organic compounds are volatile and can therefore be contained in the soil gas. In addition, the 2,4-D and the 2,4,5-T may contain dioxins such as the TCDD as by-products during the manufacturing process. For this reason, in a case in which the organic compound such as the 2,4-dichlorophenol is detected, it can be estimated that the dioxins are contained in the soil from which the soil gas is generated.

In a case in which the step (I) has the desorption treatment, the step (II) preferably has a treatment of identifying the type of the desorbed organic compound (hereinafter also referred to as a “type identification treatment”). Since the step (II) has the type identification treatment, it is possible to further improve the accuracy of determining whether or not the soil is contaminated with dioxins. The type identification treatment may be, for example, a method using a gas chromatograph mass spectrometer (GC-MS).

The concentration of the organic compound from which it can be estimated that the dioxins are contained in high concentrations (for example, 10,000 pg-TEQ/g or more), that is, the content of the organic compound from which it can be determined that the soil is contaminated with the dioxins in high concentrations, can be determined according to the concentration of the dioxins. The content of the organic compound from which it can be determined that the soil is contaminated with the dioxins in high concentrations is preferably 0.1 mg/kg or more, more preferably 1 mg/kg or more, and even more preferably 5 mg/kg or more per kg of the soil in a case in which the concentration of the dioxins is 10,000 pg-TEQ/g, for example. When the content of the organic compound is equal to or greater than the lower limit value, it is more reliably determined that the soil is contaminated with the dioxins in high concentrations. The content of the organic compound contained in the soil gas can be measured, for example, by the GC-MS.

The organic compound preferably includes one or more selected from 2,4-dichlorophenol, 2,4-dichloroanisole, 2,4,5-trichlorophenol, and 2,4,5-trichloroanisole. These organic compounds are decomposition products of the 2,4-D or the 2,4,5-T which is one of the main components of the defoliant, and the soil in which these organic compounds have been detected is highly likely to be contaminated with the dioxins in high concentrations.

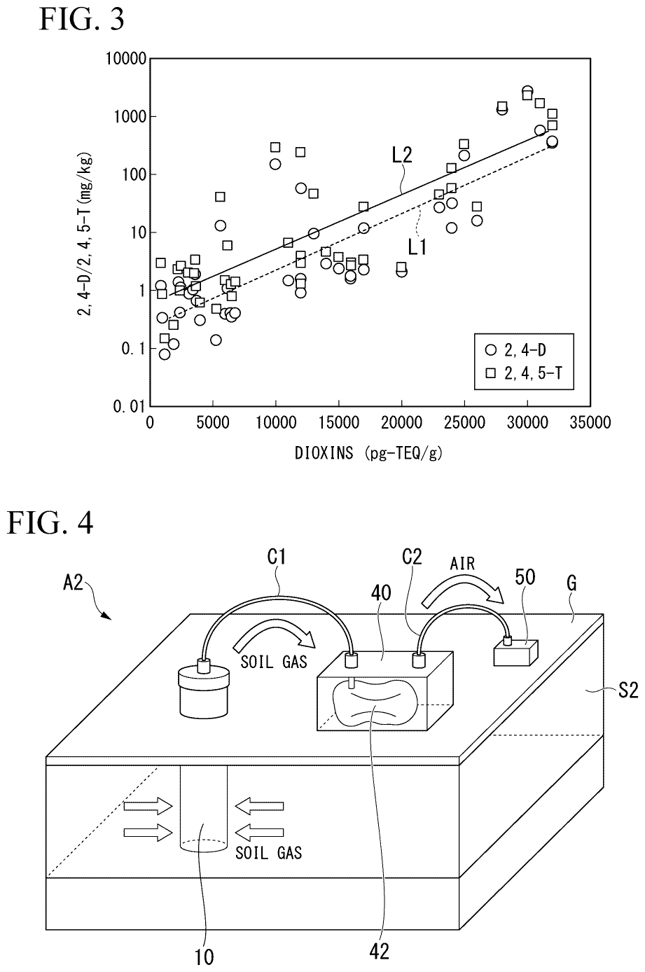

FIG. 3 illustrates the measurement results of the concentrations of the 2,4-D, the 2,4,5-T. and the dioxins in the soil contaminated with the defoliant using the GC-MS.

As shown in FIG. 3, a correlation is observed between the 2,4-D and the dioxins, and between the 2,4,5-T and the dioxins.

The correlation between the 2,4-D and the dioxins is observed as illustrated by a straight line L1.

The correlation between the 2,4,5-T and the dioxins is observed as illustrated by a straight line L2.

For example, in the soil in which the 2,4-D is contained by 10 mg/kg, it is estimated that the dioxins is detected by 17,000 pg-TEQ/g.

For example, in the soil in which the 2,4,5-T is contained by 10 mg/kg, it is estimated that the dioxins is detected by 13,000 pg-TEQ/g.

In this way, in the present process, it is possible to estimate concentrations of dioxins from the concentration of the 2,4-D or the 2,4,5-T. It is possible to obtain the concentration of the 2,4-D or the 2,4,5-T from the content of the organic compound such as the 2,4-dichlorophenol, and thus in the present process, it is possible to estimate the concentration of the dioxins contained in the soil from the content of the organic compound contained in the soil gas.

As shown in FIG. 2, in the present embodiment, the collection container 20 in which the adsorbent 30 is placed is installed inside the protective tube 10, and thus the soil gas flows from the inside of the protective tube 10 to the inside of the collection container 20. The soil gas that flows to the inside of the collection container 20 comes into contact with the adsorbent 30 and the organic compound in the soil gas is adsorbed to the adsorbent 30.

The adsorbent 30 to which the organic compound has been adsorbed is taken out, and the organic compound is desorbed, and thus it is possible to measure the concentration of the organic compound in the soil gas.

On the basis of the measured concentration of the organic compound, the concentration of the dioxins is estimated to determine whether or not the soil is contaminated with the dioxins (step (II)).

The soil contamination evaluation method of the present invention may not have the heating treatment, the adsorption treatment, and the desorption treatment. Another embodiment of the step (I), which does not has the heating treatment, the adsorption treatment, and the desorption treatment, will be described with reference to the drawing.

As shown in FIG. 4, in the case of the present embodiment, a collection hole is drilled from a ground surface G into soil S2 for an arbitrary treatment target area A2 (for example, an area of 10 m×10 m), and a protective tube 10 is buried. The upper portion of the protective tube 10 is connected to a capture bag 42 placed inside a capture box 40 through a conduit C1. The capture box 40 and a decompression pump 50 are connected through a conduit C2. The same components as those in FIG. 2 are denoted by the same reference numerals as those in FIG. 2, and the description thereof will be omitted. The size of the collection hole is similar to that of the embodiment described above.

Examples of the conduit C1 include a silicone rubber tube, a fluororubber tube, a soft polyvinyl chloride tube, and a thick-walled rubber tube. The conduit C2 may be similar to the conduit C1.

The capture box 40 may be, for example, a container made of glass or a metal. The capacity of the capture box 40 is preferably, for example, 1 to 10 liters. The capture box 40 is preferably one capable of maintaining an absolute pressure of 1 kPa or less for one hour or more.

The capture bag 42 may be, for example, a bag made of a synthetic resin film such as fluororesin or polypropylene. It is preferable that the capture bag 42 do not adsorb, permeate, or deteriorate the investigation target substance in the soil gas. The capacity of the capture bag 42 is preferably, for example, 1 to 3 liters.

A well-known decompression pump can be used as the decompression pump 50.

In step (I) in FIG. 4, first, the decompression pump 50 is operated to reduce the pressure inside the capture box 40. The suction speed when the decompression pump 50 is operated is preferably, for example, 50 to 150 mL/min. When the suction speed is within the above numerical range, it is possible to more reliably collect the soil gas.

By reducing the pressure inside the capture box 40, the capture bag 42 expands, and the soil gas inside the protective tube 10 flows into the inside of the collection bag 42 through the conduit C1.

After the soil gas has flowed into the inside of the capture bag 42, an opening portion of the capture bag 42 is tightly plugged and the capture bag 42 is taken out from the capture box 40, and thus the soil gas can be collected.

In the collected soil gas, the content of the organic compound is measured using a GC-MS or the like, the concentration of the dioxins is estimated to determine whether or not the soil is contaminated with the dioxins (step (II)).

In the soil contamination evaluation method of the present invention, the concentration of the dioxins contained in the soil is estimated from the content of the organic compound contained in the soil gas, and thus it is possible to simply and easily investigate the status of contamination of the soil contaminated with the dioxins.

In the soil contamination evaluation method of the present invention, it is possible to simply and easily investigate the status of contamination of the soil contaminated with dioxins, and thus it is possible to investigate the status of contamination of the contaminated soil without incurring much time and cost even in a wide range of the treatment target area.

In the soil contamination evaluation method of the present invention, the concentration of the dioxins contained in the contaminated soil is estimated from the content of the organic compound contained in the soil gas, and thus it is possible to investigate the status of contamination of the contaminated soil without directly collecting the contaminated soil.

When estimating the concentration of the dioxins, a correlation diagram or a calibration curve showing a relationship between the content of the organic compound contained in the soil gas and the concentration of the dioxins contained in the soil may be prepared and used.

### EXAMPLES

Hereinafter, the present invention will be described in more detail using examples, but the present invention is not limited to these examples.

### Example 1

(Capture Test of Organic Compound Volatilized from Soil)

8 g of soil (a sample) contaminated with defoliant was placed in a 20 mL glass vial, and an adsorbent (silica gel with continuous pores and fine pores in a silica skeleton) was suspended in the upper gas phase, then the glass vial was tightly plugged and left standing in a thermostatic chamber set at 60° C. for 60 minutes to allow the organic compound in the soil gas to be adsorbed to the adsorbent (a capture test).

At the same time, the following blank test was performed to check the adsorption of the organic compound (derived from a blank) during the capture.

### Container Blank

With respect to an empty test container (20 mL glass vial) in which the sample was not placed, a capture test was performed in the same manner as for the sample (60° C., 60 minutes).

### Laboratory Blank

The adsorbent was not placed in a test container and was directly exposed to the laboratory atmosphere during a capture test. The temperature of the laboratory atmosphere was 27.7° C. and the exposure time was 120 minutes.

The organic compound adsorbed to the adsorbent was thermally desorbed (250° C., 1.5 minutes) using a portable thermal desorber (Handy TD TD265 manufactured by GL Sciences Inc.), and the entire amount was introduced into a GC-MS.

The GC-MS analysis conditions are as follows.
Model name: Agilent 6890/5975
Separation column: DB-WAX 60 m×25 mmφ, film thickness 0.25 μm
Column temperature conditions: 40° C. (retention for 3 min)→10° C./min→100° C.→3° C./min→250° C. (retention for 5 min)
Carrier gas: Helium 1.0 mL/min
Data acquisition method: SCAN mode (measurement range m/z 35 to 550)

### Example 2

The capture test was performed in the same manner as in Example 1 except that the test container was left standing in the thermostatic chamber set at 60° C. for 10 minutes.

### Example 3

The capture test was performed in the same manner as in Example 1 except that the test container was left standing at room temperature (27.7° C.) for 120 minutes.

### Example 4

The capture test was performed in the same manner as in Example 3 except that the test container was left standing at room temperature (27.7° C.) for 30 minutes.

In each example, the time it took for each component of the gas introduced into the GC-MS to reach the detector (retention time) was measured, and a qualitative analysis was performed. The results are shown in Tables 1 and 2.

In the table, “o” indicates that the component was detected, and “-” indicates that the component was not detected. The organic compound names shown in the table exclude the components detected in the blank test. In addition, the table does not list the compound names of some of the detected components, including substances whose structures cannot be identified, various hydrocarbons, and the same compound with different retention times. In addition, for organic compounds that have isomers, the identification of the isomers were not performed.

TABLE 1

| Retention | | | | | |
|---|---|---|---|---|---|
| time | | Example | Example | Example | Example |
| (min) | Organic compound name | 1 | 2 | 3 | 4 |
| 6.259 | Siloxane | ∘ | — | — | — |
| 11.012 | Methylbornene | ∘ | ∘ | ∘ | ∘ |
| 20.563 | Pentadecane | — | ∘ | ∘ | ∘ |
| 20.586 | Pentadecane | ∘ | — | — | — |
| 24.032 | Methylisoborneol | — | ∘ | ∘ | ∘ |
| 24.054 | Methylisoborneol | ∘ | — | — | — |
| 25.856 | Methylpropylmethoxybenzene | ∘ | — | — | — |
| 32.118 | Geosmin | ∘ | — | — | — |
| 32.637 | Methylnaphthalene | — | ∘ | — | — |
| 32.659 | Methylnaphthalene | — | — | ∘ | ∘ |
| 32.682 | Methylnaphthalene | ∘ | — | — | — |
| 33.600 | Tetrachloropyridine | ∘ | — | — | — |
| 33.583 | Dichloroanisole | — | ∘ | — | — |
| 33.650 | Dichloroanisole | ∘ | — | — | — |
| 33.830 | Methylnaphthalene | — | ∘ | — | — |
| 33.853 | Methylnaphthalene | ∘ | — | — | — |
| 34.056 | Dichloroanisole | — | ∘ | — | — |
| 34.078 | Dichloroanisole | ∘ | — | — | ∘ |
| 34.100 | Dichloroanisole | — | — | ∘ | — |
| 34.866 | Dichlorophenol alkyl ester | ∘ | ∘ | — | — |
| 35.790 | Ethylnaphthalene | ∘ | — | — | — |
| 36.331 | Dimethylnaphthalene | ∘ | ∘ | — | — |
| 37.119 | Trimethylnaphthalene | ∘ | — | — | — |
| 37.525 | Dimethylnaphthalene | — | ∘ | — | — |
| 37.569 | Dimethylnaphthalene | ∘ | — | — | — |

TABLE 2

| Retention | | | | | |
|---|---|---|---|---|---|
| time | | Example | Example | Example | Example |
| (min) | Organic compound name | 1 | 2 | 3 | 4 |
| 38.200 | Trichlorophenol alkyl ester | ∘ | ∘ | — | — |
| 39.214 | Dichlorophenol alkyl ester | ∘ | ∘ | — | — |
| 39.236 | Dichlorophenol alkyl ester | ∘ | ∘ | — | — |
| 39.867 | Trichloroethoxybenzene | ∘ | ∘ | — | — |
| 40.588 | Trichloroanisole | — | ∘ | ∘ | ∘ |
| 40.610 | Trichloroanisole | ∘ | — | — | — |
| 42.029 | Trichlorophenol alkyl ester | ∘ | — | — | — |
| 42.728 | Dichlorophenol | — | ∘ | ∘ | ∘ |
| 42.750 | Dichlorophenol | ∘ | — | — | — |
| 43.854 | Dimethylbiphenyl | ∘ | — | — | — |
| 44.200 | Tetramethylnaphthalene | ∘ | — | — | — |
| 44.800 | Trichlorophenol alkyl ester | — | — | ∘ | ∘ |
| 44.845 | Trichlorophenol alkyl ester | — | ∘ | — | — |
| 44.868 | Trichlorophenol alkyl ester | ∘ | — | — | — |
| 45.341 | Dichloromethoxybenzene | — | — | ∘ | — |
| 45.363 | Dichloromethoxybenzene | ∘ | — | — | — |
| 45.498 | Hexachlorobenzene | ∘ | — | — | — |
| 46.692 | Butylphenol | ∘ | ∘ | — | — |
| 46.827 | Dibutylphenol | ∘ | — | — | — |
| 47.751 | Dichlorodimethoxybenzene | ∘ | ∘ | — | — |
| 52.400 | Dodecanoic acid | ∘ | ∘ | ∘ | — |
| 52.954 | Trichlorophenol | ∘ | ∘ | — | — |
| 56.648 | Trichlorophenol | ∘ | ∘ | — | — |

As shown in Tables 1 and 2, many organic compounds containing chlorine in their structures were detected as the organic compounds derived from the samples. Among these, trichloroanisole was detected with a high intensity and was a characteristic organic compound. Since the trichloroanisole is produced by the decomposition of 2,4,5-T in the environment, it is estimated that there is a high possibility that the contaminated soil used in Examples 1 to 4 was contaminated with dioxins. In other words, it is estimated that the dioxins are contained in the contaminated soil in potentially harmful concentrations.

As other organic compounds that may be derived from the defoliant, dichloroanisole, dichlorophenol, trichlorophenol, methylnaphthalene, and the like was detected.

Regarding the conditions for capturing the organic compounds, it was checked that more organic compounds were detected at a higher temperature (60° C.) than at the room temperature (27.7° C.). In addition, it was also checked that, at the same temperature, more organic compounds were detected with a longer time (60 minutes or 120 minutes) than with a shorter time (10 minutes or 30 minutes).

From the above results, it was found that according to the soil contamination evaluation method for the contaminated soil of the present invention, it is possible to simply and easily investigate the status of contamination of soil contaminated with dioxins.

17 international goals adopted at the united nations summit in September 2015 are “sustainable development goals (SDGs).” The soil contamination evaluation method for the contaminated soil according to the present embodiment can contribute to the achievement of, for example, goal “11. Sustainable cities and communities” out of the 17 goals of the SDGs.

### REFERENCE SIGNS LIST

10 Protective tube
20 Collection container
30 Adsorbent
40 Capture box
42 Capture bag
50 Decompression pump
A1, A2 Treatment target area
S1, S2 Soil
G Ground surface
C1, C2 Conduit

1. A soil contamination evaluation method comprising:
a step (I) of collecting soil gas from soil; and
a step (II) of estimating concentrations of dioxins contained in the soil from a content of an organic compound contained in the soil gas.

2. The soil contamination evaluation method according to claim 1, wherein in the step (II), it is determined that the soil is contaminated with the dioxins in high concentrations when the content of the organic compound is 0.1 mg/kg or more.

3. The soil contamination evaluation method according to claim 1,
wherein the step (I) has a treatment of adsorbing the organic compound to an adsorbent and a treatment of desorbing the organic compound from the adsorbent to which the organic compound has been adsorbed, and
wherein the step (II) has a treatment of identifying a type of the desorbed organic

4. The soil contamination evaluation method according to claim 1, wherein the step (I) has a treatment of heating the soil.

5. The soil contamination evaluation method according to claim 1, wherein the organic compound includes one or more selected from 2,4-dichlorophenol, 2,4-dichloroanisole, 2,4,5-trichlorophenol, and 2,4,5-trichloroanisole.

6. The soil contamination evaluation method according to claim 2,
wherein the step (I) has a treatment of adsorbing the organic compound to an adsorbent and a treatment of desorbing the organic compound from the adsorbent to which the organic compound has been adsorbed, and
wherein the step (II) has a treatment of identifying a type of the desorbed organic

7. The soil contamination evaluation method according to claim 2, wherein the step (I) has a treatment of heating the soil.

8. The soil contamination evaluation method according to claim 2, wherein the organic compound includes one or more selected from 2,4-dichlorophenol, 2,4-dichloroanisole, 2,4,5-trichlorophenol, and 2,4,5-trichloroanisole.
